(19) United States
(12) Patent Application Publication
(10) Pub. No.: US 2021/0251882 A1
(43) Pub. Date: Aug. 19, 2021

(54) TOPICAL COSMETIC COMPOSITIONS

(71) Applicant: Mary Kay Inc., Addison, TX (US)

(72) Inventors: Geetha KALAHASTI, Argyle, TX (US); Doug ZUBER, Frisco, TX (US)

(21) Appl. No.: 17/248,969

(22) Filed: Feb. 16, 2021

Related U.S. Application Data
(60) Provisional application No. 62/977,631, filed on Feb. 17, 2020.

(51) Int. Cl. A61K 8/9789; A61K 8/60; A61K 8/55; A61K 8/96; A61K 8/64; A61Q 19/08; A61K 8/06; A61Q 19/02

(52) U.S. Cl. CPC A61K 8/9789; A61K 8/602; A61K 8/553; A61Q 19/02; A61K 8/64; A61Q 19/08; A61K 8/062; A61K 8/96

(57) ABSTRACT

The present invention relates generally to methods and compositions useful for a topical composition comprising escin, lecithin, plankton extract, Hieracium pilosella extract, Bellis perennis flower extract, hesperidin methyl chalcone, palmitoyl tetrapeptide-7, Rosmarinus officinalis leaf extract, or any combination thereof and methods of use and production thereof.

### CROSS-REFERENCE TO RELATED APPLICATIONS

The present application claims priority to U.S. provisional patent application 62/977,631, filed Feb. 17, 2020, the disclosure of which is hereby incorporated by reference in its entirety.

### BACKGROUND OF THE INVENTION

### A. Field of the Invention

The present invention relates generally to topical compositions that can be used to improve skin conditions and/or visual appearance of skin. In certain aspects, the compositions of the present invention can include, for example, a combination of ingredients to increase skin firmness, reduce the appearance of a fine line or wrinkle of skin, under-eye puffiness, and/or dark circles on the skin around an eye area. This combination of ingredients can be included in a wide-range of product formulations (e.g., serums, eye creams, toners, gels, masks, etc.).

### B. Description of Related Art

Ageing, chronic exposure to adverse environmental factors, malnutrition, fatigue, etc., can change the visual appearance, physical properties, or physiological functions of skin in ways that are considered visually undesirable. The most notable and obvious changes include the development of fine lines and wrinkles, loss of elasticity, increased sagging, loss of firmness, loss of color evenness or tone, coarse surface texture, and mottled pigmentation. Less obvious but measurable changes which occur as skin ages or endures chronic environmental insult include a general reduction in cellular and tissue vitality, reduction in cell replication rates, reduced cutaneous blood flow, reduced moisture content, accumulated errors in structure and function, alterations in the normal regulation of common biochemical pathways, and a reduction in the skin's ability to remodel and repair itself. Many of the alterations in appearance and function of the skin are caused by changes in the outer epidermal layer of the skin, while others are caused by changes in the lower dermis.

Many factors contribute to skin aging and the appearance of aging such as the actual age of a person, the amount of exposure to environmental factors (e.g., sun light, pollution, chemicals, smoke, etc.), and how well a person has taken care of their skin. In particular, skin aging concerns two processes—intrinsic aging, which is related to the natural aging process and genetic influences, and extrinsic aging, which is accumulated damage due to environmental factors.

Intrinsic aging process in cells and skin can be related to the loss of proper function of the skin in maintaining biochemical pathways. Such pathways can control the oxidative/reductive environment balance in the skin, the regulation of cell division and cellular membrane integrity, and the maintenance of the moisture balance of the skin. The loss of the proper function of the skin can lead to loss of skin firmness, increased skin unevenness, increased fine lines and wrinkles, and dry skin. As one example, intrinsic aging can be due to the function of the protein Lamin A, which is important during cell division as it provides the membrane structure of the nuclease. Without functional Lamin A, the nuclear lamina creates an abnormal nuclear envelope lacking structural support. This can lead to an abnormal shaped nuclear envelope which limits cell division. A muted form of Lamin A, known as progerin, is associated with the disease progeria where patients suffer from accelerated aging, displaying signs of aging in skin as early as 2 years of age, and have a sharply shortened lifespan. This, and other losses of proper function of the skin can lead to loss of skin firmness, increased skin unevenness, increased fine lines and wrinkles, increased oxidative damage, and dry skin.

Extrinsic factors can include exposure to ultraviolet (UV) rays, irritants, and pollution. UV rays, through sun exposure or the use of ultraviolet lamps (for example, tanning beds), can induce oxidative stress, inflammation, production of melanin, and even genetic mutations that leads to skin damage.

The accumulation of oxidative stress through free radical formation, can damage skin proteins leading to skin aging, which includes loss of elasticity, loss of dermal proteins, lines and wrinkles, and abnormal pigmentation.

Inflammation is also a characteristic of UV and environmental damage. Inflammation can occur through inflammatory cytokines such as TNF-α or VEGF, or enzymes that contribute to the inflammatory pathway. As inflammation persists, enzymes such as matrix metalloproteinase-1 (MMP1), matrix metalloproteinase-3 (MMP3), and matrix metalloproteinase-9 (MMP9) are involved in the breakdown of dermal proteins, which allows immune cells to migrate. This breakdown in dermal proteins such as laminin, elastin, and collagen can lead to skin aging.

When exposed to extrinsic factors, the keratinocyte (outermost cell of the skin) releases signaling molecules, such as α-melanocyte-stimulating hormone (α-MSH), and inflammatory cytokines. These hormones trigger melanocytes to produce melanin (melanogenesis). Typical pigmentation is characterized by an even, uniform coloration of the skin. However, the production of melanin through extrinsic factors can result in variations in the color of the skin. For example, a person's skin can have a sallow tone, hyperpigmented spots, unwanted freckles or dark spots such as senile lentigo, liver spots, melasma, brown or age spots, vitiligo, sunburn pigmentation, post-inflammatory hyperpigmentation due to abrasion, burns, wounds or dermatitis, phototoxic reaction and other similar small, fixed pigmented lesions. It is often desirable to lighten these areas or even out the appearance of irregularly pigmented areas of skin. Individuals may also wish to increase fairness or reduce the overall level of pigmentation in the skin. In either case, the hyperpigmentation is usually viewed as cosmetically undesirable and individuals often wish to lighten the skin. Conventional depigmenting agents, such as hydroquinone, corticosteroids, and kojic acid can raise several safety concerns (for example, ochronosis, atrophy, carcinogenesis, and other local or systemic side effects) with long-term exposure. (Talwar 1993).

Extrinsic factors can also reduce the moisture in skin. Exposure to chemicals, solvents, washing, cosmetics, fabrics, or dry environments are some of the many ways that skin can lose moisture. Loss of moisture can lead to breaks or fine lines and wrinkles in the skin. Maintaining moisture of the skin and/or hair helps overcome some unwanted changes in skin and hair. However, maintaining moisture of the skin can be difficult in the daily exposure to harsh extrinsic factors. Moisturizers have been designed to attempt to remedy such difficulties. Moisturizers for the skin and hair are complex mixtures of chemical agents specially designed to make the external layers of the skin (epidermis) softer and more pliable. They increase the skin's hydration (water content) by reducing evaporation. Naturally occurring skin lipids and sterols, as well as artificial or natural oils, humectants, emollients, lubricants, etc., may be part of the composition of commercial skin moisturizers. They usually are available as commercial products for cosmetic and therapeutic uses, but can also be made at home using common pharmacy ingredients. However, moisturizers are not perfect. Some problems associated with moisturizers include unpleasant tactile properties (e.g., heavy, greasy, or sticky feel), instability, skin-irritation, or insufficient moisturization capabilities.

The combination of intrinsic and extrinsic factors eventually leads to visible signs of aging. Current products on the market either do not effectively address the signs or causes of ageing or the effects of extrinsic factors on the skin and/or they have skin irritating effects. For example, current products may not address loss of skin firmness, pigmentation problems, appearance of fine lines or wrinkles, and/or loss of moisture.

### SUMMARY OF THE INVENTION

A solution to the problems associated with current products to counteract some of the effects of ageing and exposure to extrinsic factors that change the appearance and/or condition of skin has been discovered. The solution resides in a combination of ingredients, which may be any one of or any combination of escin, lecithin, plankton extract, hieracium pilosella extract, bellis perennis flower extract, hesperidin methyl chalcone, palmitoyl tetrapeptide-7, and Rosmarinus officinalis leaf extract, that can be used to create a topical skin composition to effectively increase skin firmness and elasticity, reduce skin lines and/or wrinkles, reduce under-eye puffiness, under-eye dark circles, and/or saggy eyelids, to improve the overall appearance of the area around the eye, and to increase moisture of the skin and/or hair. Benefits of the topical skin composition include: increasing lysyl oxidase, increasing laminin, inhibiting elastase, inhibiting MMP1, MMP9, and MMP3, inhibiting pro-inflammatory cytokines including lipoxygenase, IL-6, 11-8, TNF-alpha, FAAH, inhibiting elastatse, inhibiting renin, inhibiting Melanogenesis, and providing antioxidant capacity.

In some embodiments, there is disclosed a topical skin composition. In some instances, the topical skin composition can include any one of, any combination of, or all of escin, lecithin, plankton extract, hieracium pilosella extract, bellis perennis flower extract, hesperidin methyl chalcone, palmitoyl tetrapeptide-7, and Rosmarinus officinalis leaf extract. The amounts of the ingredients within the composition can vary (e.g., amounts can be as low as 0.000001% to as high as 98% w/w or any range therein). In some instances the composition includes 0.01% to 1% by weight of escin, 0.01% to 1% by weight of lecithin, 0.01% to 1% by weight of plankton extract, 0.1 to 10 wt. % hieracium pilosella extract, 0.1 to 10 wt. % bellis perennis flower extract, 0.001 to 0.1 wt. % hesperidin methyl chalcone, 0.001 to 0.01 wt. % palmitoyl tetrapeptide-7, 0.001 to 0.1 wt. % Rosmarinus officinalis leaf extract. In some aspects, the escin may comprise a mixture of saponins obtained from seeds of horse chestnut tree. The lecithin may be a moisturizer that is mixed with escin. The plankton extract may include an exopolysaccharide synthesized by a micro-organism called vibrio algenolyticus that belongs to the family of thalassoplankton. In some aspects, the hieracium pilosella extract and the bellis perennis flower extract may include a synergistic combination of extracts of hieracium pilosella and bellis perennis flower. In a preferred instance, the Rosmarinus officinalis leaf extract can be obtained from the leaf of Rosmarinus officinalis. The leaf can be subjected to an eutectigenesis extraction process using a fluid extraction mixture comprising betaine or hydrated betaine, a hydrogen bond donor compound (e.g., polyols, organic acids, etc.), and water. In some instances, the composition further comprises water. In one instance, the composition comprises 35% to 70% by weight of water. In some aspects, the composition further comprises glycerin, disodium EDTA, xanthan gum, stearic acid, squalene, cetyl alcohol, mineral oil, ceteareth-25, disodium ethylene dicocamide PEG-15 disulfate, bees wax, octyl dodecnol, stearyl alcohol, ammonium acryloyldimethyltaurate/VP copolymer, paraffin, microcrystalline wax, isohexadecane, capryl methicone, dimethicone, and dimethiconol. In some instances, the composition can comprise 1 to 20% by weight of glycerin, 0.01 to 10% by weight of disodium EDTA, 0.01 to 1% by weight of xanthan gum, 0.1 to 20% by weight of stearic acid, 0.2 to 10% by weight of squalene, 0.3 to 10% by weight of cetyl alcohol, 0.1 to 10% by weight of mineral oil, 0.1 to 10% by weight of ceteareth-25, 0.1 to 10% by weight of disodium ethylene dicocamide PEG-15 disulfate, 0.1 to 5% by weight of bees wax, 0.1 to 10% by weight of octyl dodecanol, 0.1 to 10% stearyl alcohol, 0.1 to 10% by weight of ammonium acryloyldimethyltaurate/VP copolymer, 0.01 to 1% by weight of paraffin, 0.1 to 10% by weight of microcrystalline wax, 0.1 to 10% by weight of isohexadecane, 0.1 to 10% by weight of caprylyl methicone, 0.1 to 20% by weight of dimethicone, 0.1 to 10% by weight of dimethiconol. In some aspects, the composition can further comprise cyclopentasiloxane, cyclohexasiloxane, triethanolamine, beta-sitosterol, diazolidinyl urea, hydroxypropyl cyclodextrin, iodopropynyl butylcarbamate, lactic acid, betaine, chlorhexidine digluconate, dipeptide-2, and potassium sorbate. In some instances, the composition can comprise 0.001 to 0.1% by weight of cyclopentasilooxane, 0.001 to 0.1% by weight of cyclohexasiloxane, 0.1 to 10% by weight of triethanolamine, 0.01 to 1% by weight of beta-sitosterol, 0.01 to 3% by weight of diazolidinyl urea, 0.01 to 1% by weight of hydroxypropyl cyclodextrin, 0.001 to 0.5% by weight of iodopropynyl butylcarbamate, 0.01 to 1% by weight of lactic acid, 0.01 to 1% by weight of betaine, 0.0001 to 0.01% by weight of chlorhexidine digluconate, 0.0001 to 0.01% by weight of dipeptide-2, 0.0001 to 0.01% by weight of potassium sorbate, 0.00001 to 0.01% by weight of palmitoyl tetrapeptide-7. In some aspects, the composition can further comprise butylene glycol, opuntia tuna fruit extract, citric acid, sodium benzoate, silica, titanium dioxide, MICA, tin oxide, and denatured alcohol. In some instances, the composition can comprise 0.1 to 10% by weight butylene glycol, 0.00001 to 0.01% by weight of opuntia tuna fruit extract, 0.00001 to 0.001% by weight of citric acid, 0.00001 to 0.001% by weight of sodium benzoate, 0.1 to 10% by weight of silica, 0.01 to 5% by weight of titanium dioxide, 0.01 to 5% by weight of MICA, 0.001 to 0.1% by weight of tin oxide, and 0.1 to 10% by weight of denatured alcohol. The composition may further comprise one or more ingredients described herein. For example, the composition may comprise one or more additional ingredients selected from one or more conditioning agents, moisturizing agents, pH adjusters, structuring agents, inorganic salts, and preservatives.

In some aspects, the topical composition can include an effective amount of Hieracium pilosella extract and Bellis perennis flower extract to increase lysyl oxidase. In some aspects, the topical composition may comprise an effective amount of hesperidin methyl chalcone and palmitoyl tetrapeptide-7 to increase laminin expression. In some aspects, the topical composition can comprise an effective amount of Rosmarinus officinalis leaf extract to inhibit Matrix Metalloproteinase Enzyme Activity. Exemplary Matrix Metalloproteinase Enzyme can include MMP1, MMP3, MMP9, or any combination thereof. In some aspects, the topical composition can include an effective amount of Rosmarinus officinalis leaf extract to inhibit a pro-inflammatory cytokine. In some instances, the pro-inflammatory cytokine can include lipoxygenase, IL-6, 11-8, TNF-alpha, FAAH, or any combination thereof. In some aspects, the topical composition may include an effective amount of escin to inhibit a pro-inflammatory cytokine including FAAH. In some aspects, the topical composition may include an effective amount of Rosmarinus officinalis leaf extract to inhibit elastase. The topical composition can include an effective amount of hesperidin methyl chalcone and palmitoyl tetrapeptide-7 to inhibit renin. In some aspects, the topical composition can include an effective amount of escin to inhibit melanogenesis. In some aspects, the topical composition may be capable of providing antioxidant capacity to the skin. The topical composition can be capable of reducing lines and/or wrinkles of skin. In some aspects, the topical composition is capable of improving skin firmness. In some instances, the topical composition can be capable of improving saggy skin around an eye area and/or reducing under-eye puffiness. In some aspects, the topical composition can be capable of reducing dark circles around an eye area.

In some aspects, topical composition can include an emulsion, a serum, a gel, a gel emulsion, or a gel serum. In some aspects, the topical skin composition can include an emulsion. In one instance, the composition is an oil-in-water emulsion. In some instances, the composition may be formulated as a cream. In yet another instance, the composition is formulated as an eye cream.

Methods of use for the topical compositions are also disclosed. In some embodiments, a method of improving a condition or appearance of skin and/or hair is disclosed. The method can comprise applying to skin an effective amount of a topical composition comprising escin, lecithin, plankton extract, hieracium pilosella extract, bellis perennis flower extract, hesperidin methyl chalcone, palmitoyl tetrapeptide-7, Rosmarinus officinalis leaf extract, or any combination thereof. In one aspect, the topical compositions disclosed are applied to skin and/or hair and left on the skin and/or hair, or alternatively removed from the skin and/or hair after a period of time. In some instances, after topical application, the topical composition may remain on the skin for at least 30 minutes. In other aspects, by applying the topical composition, the skin can be treated to increase skin firmness, wherein the firmness of the skin is increased. In yet some other aspects, by applying the topical composition, the skin can be treated to reduce the appearance of a fine line and/or wrinkle, wherein the appearance of a fine line and/or a wrinkle of the skin is reduced. In other aspects, by applying the topical composition to skin of an eye area, the skin is treated to reduce under-eye puffiness, wherein under-eye puffiness is reduced. In yet other aspects, by applying the topical composition, the skin can be treated to improve saggy skin, wherein skin sagging is reduced. In some other aspects, by applying the topical composition, the skin can be treated to improve firmness and/or elasticity and/or reduce dark circles around eye area of skin, wherein skin firmness and/or skin elasticity are improved, and/or dark circles around skin of eye area of are reduced.

In some aspects, the topical compositions disclosed can be adapted to inhibit melanogenesis in skin by applying any one of the topical compositions disclosed herein to skin, wherein melanogenesis is inhibited. In yet another aspect, the topical compositions disclosed can be adapted to reduce a dark circle on and/or under the skin around the eye by applying any one of the compositions disclosed herein to skin around the eye, wherein the dark circle on and/or under the skin is reduced. In some aspects, the compositions disclosed can be adapted to improve saggy eyelids by applying any one of the compositions disclosed herein to skin around eye, wherein the saggy eyelids are reduced. In another aspect, the composition disclosed here are used to reduce lines and wrinkles on skin around the eye by applying any one of the compositions disclosed herein to skin around eye, wherein the lines and wrinkles are reduced. In yet another aspect, the topical compositions disclosed can be adapted to improve skin firmness by applying any one of the compositions disclosed herein to skin, including skin around the eye, wherein the skin firmness is improved. In another aspect, the compositions disclosed are adapted to reduce dark circles around eyes by applying any one of the topical compositions disclosed herein to skin around eyes, wherein the dark circles are reduced. In another aspect, the compositions disclosed herein are used to improve the overall appearance of skin around the eye by applying any one of the compositions disclosed herein to skin around the eye, wherein the overall appearance of skin around the eye is improved. In some aspect, the topical composition that is applied to the skin may include an effective amount of hieracium pilosella extract and bellis perennis flower extract to increase lysyl oxidase, and the topical compositions are used to increase lysyl oxidase. In yet some other aspects, the topical composition that is applied to the skin can include an effective amount of hesperidin methyl chalcone and palmitoyl tetrapeptide-7 to increase laminin expression, and the topical compositions can be used to increase laminin expression. In some other aspects, the topical composition that is applied to the skin may include an effective amount of Rosmarinus officinalis leaf extract to inhibit Matrix Metalloproteinase Enzyme Activity, and the topical composition is used to inhibit Matrix Metalloproteinase Enzyme Activity. Exemplary Matrix Metalloproteinase Enzyme can include MMP1, MMP3, MMP9, or any combination thereof. In some aspects, the topical composition that is applied to the skin can include an effective amount of Rosmarinus officinalis leaf extract to inhibit elastase, and the topical composition is used to inhibit elastase. In some other aspects, the topical composition that is applied to the skin can include an effective amount of a combination of hesperidin methyl chalcone and palmitoyl tetrapeptide-7 to inhibit renin, and the topical composition is used to inhibit renin. In some aspects, the topical composition that is applied to the skin can include an effective amount of escin to inhibit melanogenesis, and the topical composition is used to inhibit melanogenesis. In some other aspects, the topical composition that is applied to the skin can include an effective amount of Rosmarinus officinalis leaf extract to inhibit a pro-inflammatory cytokine, and the topical composition is used to inhibit the pro-inflammatory cytokine. Exemplary pro-inflammatory cytokines include lipoxygenase, IL-6, 11-8, TNF-α, FAAH, or any combinations thereof. In some other aspects, the topical composition that is applied to the skin includes an effective amount of escin to inhibit a pro-inflammatory cytokine including FAAH. In some aspects, the topical composition that is applied to the skin is adapted to provide antioxidant capacity. In some aspects, the topical composition is applied to skin around an eye area. In other some aspects, the topical composition is applied to fine line(s) and/or wrinkle(s) on the skin. In some aspects, the topical composition is applied to dark circle of skin in an eye area. In some instances, the topical composition is applied to saggy skin and/or non-elastic skin. In some aspects, after topical composition is applied on the skin for at least 30 minutes. In some instances, the topical composition is applied daily for at least 4 weeks.

In particular aspects, the compositions of the present invention are formulated as a topical skin and/or hair composition. The composition can have a dermatologically acceptable vehicle or carrier for the compounds, compositions, and extracts. The composition can further include a moisturizing agent or a humectant, a surfactant, a silicone containing compounds, a UV agent, an oil, and/or other ingredients identified in this specification or those known in the art. The composition can be a lotion, cream, gel, serum, emulsion (e.g., oil-in-water, water-in-oil, silicone-in-water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, etc. The composition can be in powdered form (e.g., dried, lyophilized, particulate, etc.). The composition can be formulated for topical skin application at least 1, 2, 3, 4, 5, 6, 7, or more times a day during use. In other aspects of the present invention, compositions can be storage stable or color stable, or both. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

The compositions of the present invention can also be modified to have a desired oxygen radical absorbance capacity (ORAC) value. In certain non-limiting aspects, the compositions of the present invention or the component or extracts thereof identified throughout this specification can be modified to have an ORAC value per mg of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 95, 100, 200, 300, 400, 500, 600, 700, 800, 900, 1000, 2000, 3000, 4000, 5000, 6000, 7000, 8000, 9000, 10000, 15000, 20000, 30000, 50000, 100000 or more or any range derivable therein.

The compositions in non-limiting aspects can have a pH of about 6 to about 9. In other aspects, the pH can be 1, 2, 3, 4, 5, 6, 7, 8, 9, 10, 11, 12, 13, or 14. The compositions can include a triglyceride. Non-limiting examples include small, medium, and large chain triglycerides. In certain aspects, the triglyceride is a medium chain triglyceride (e.g., caprylic capric triglyceride). The compositions can also include preservatives. Non-limiting examples of preservatives include methylparaben, propylparaben, or a mixture of methylparaben and propylparaben. In some embodiments, the preservative is not a paraben.

Compositions of the present invention can have UVA and UVB absorption properties. The compositions can have an sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions can be sunscreen lotions, sprays, or creams.

The compositions of the present invention can also include any one of, any combination of, or all of the following additional ingredients: water, a chelating agent, a moisturizing agent, a preservative, a thickening agent, a silicone containing compound, an essential oil, a structuring agent, a vitamin, a pharmaceutical ingredient, or an antioxidant, or any combination of such ingredients or mixtures of such ingredients. In certain aspects, the composition can include at least two, three, four, five, six, seven, eight, nine, ten, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present invention are also contemplated. In certain embodiments, the composition is comprised in a container. The container can be a bottle, dispenser, or package. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

It is also contemplated that the compositions disclosed throughout this specification can be used as a leave-on or rinse-off composition. By way of example, a leave-on composition can be one that is topically applied to skin and remains on the skin for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the skin and then removed or rinsed from the skin (e.g., with water) within a period of time such as less than 5, 4, 3, 2, or 1 minute. An example of a rinse of composition can be a skin cleanser, shampoo, conditioner, or soap. An example of a leave-on composition can be a skin moisturizer, sunscreen, mask, overnight cream, or a day cream.

It is contemplated that any embodiment discussed in this specification can be implemented with respect to any method or composition of the invention, and vice versa. Furthermore, compositions of the invention can be used to achieve methods of the invention.

In one embodiment, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

Also contemplated is a product comprising a composition of the present invention. In non-limiting aspects, the product can be a cosmetic product. The cosmetic product can be those described in other sections of this specification or those known to a person of skill in the art. Non-limiting examples of products include a moisturizer, a cream, a lotion, a skin softener, a gel, a wash, a foundation, a night cream, a lipstick, a cleanser, a toner, a sunscreen, a mask, an anti-aging product, a deodorant, an antiperspirant, a perfume, a cologne, etc.

In the context of the present invention, at least the following 40 aspects are described. Aspect 1 includes a method of improving a condition or appearance of skin. The method comprising applying to the skin an effective amount of a topical composition comprising escin, lecithin, plankton extract, hieracium pilosella extract, bellis perennis flower extract, hesperidin methyl chalcone, palmitoyl tetrapeptide-7, Rosmarinus officinalis leaf extract, or any combination thereof. Aspect 2 depends on aspect 1, wherein, by applying the topical composition, the skin is treated to reduce a skin condition comprising skin lines, skin wrinkles, under-eye puffiness, under-eye dark circles, saggy eyelids, saggy skin, or any combination thereof. Aspect 3 depends on any of aspects 1 and 2, wherein, by applying the topical composition, the skin is treated to improve skin firmness and/or skin elasticity, and/or increase moisture of the skin. Aspect 4 depends on any of claims 1 to 3, wherein, by applying the topical composition, the skin is treated to inhibit Matrix Metalloproteinase Enzyme Activity. Aspect 5 depends on aspect 4, wherein the Matrix Metalloproteinase Enzyme includes MMP1, MMP3, MMP9, or any combination thereof. Aspect 6 depends on any of aspects 1 to 5, wherein, by applying the topical composition, the skin is treated to stimulate laminin production. Aspect 7 depends on any of aspects 1 to 6, wherein, by applying the topical composition, the skin is treated to increase lysyl oxidase. Aspect 8 depends on any of aspects 1 to 7, wherein, by applying the topical composition, the skin is treated to inhibit elastase, renin, melanogenesis, or any combination thereof. Aspect 9 depends on any of aspects 1 to 8, wherein, by applying the topical composition, the skin is treated to inhibit a pro-inflammatory cytokine. Aspect 10 depends on aspect 9, wherein the pro-inflammatory cytokine includes lipoxygenase, IL-6, 11-8, TNF-alpha, FAAH, or any combination thereof. Aspect 11 depends on any of aspects 1 to 10, wherein the topical composition is adapted to provide antioxidant capacity. Aspect 12 depends on any of aspects 1 to 11, wherein the topical composition further comprises water. Aspect 13 depends on any of aspects 1 to 12, wherein the topical composition is a cream, an emulsion, a serum, a gel, a gel emulsion, or a gel serum. Aspect 14 depends on any of aspects 1 to 13, wherein the topical composition is an oil-in-water emulsion. Aspect 15 depends on any of aspects 1 to 14, wherein the topical composition is applied to skin around an eye area. Aspect 16 depends on any of aspects 1 to 15, wherein the topical composition is applied to a fine line or a wrinkle on the skin. Aspect 17 depends on of any of aspects 1 to 16, wherein the topical composition is applied to a dark circle of skin in an eye area. Aspect 18 depends on any of aspects 1 to 17, wherein the topical composition is applied to saggy skin or non-elastic skin. Aspect 19 depends on any of aspects 1 to 18, wherein, after topical application, the composition remains on the skin for at least 30 minutes. Aspect 20 depends on any of aspects 1 to 19, wherein the topical composition is applied daily for at least 4 weeks. Aspect 21 includes a topical skin composition comprising escin, lecithin, plankton extract, hieracium pilosella extract, bellis perennis flower extract, hesperidin methyl chalcone, palmitoyl tetrapeptide-7, Rosmarinus officinalis leaf extract, or any combination thereof. Aspect 22 depends on aspect 21, wherein the topical composition comprises an effective amount of hieracium pilosella extract and bellis perennis flower extract to increase lysyl oxidase. Aspect 23 depends on any of aspects 21 and 22, wherein the topical composition comprises an effective amount of hesperidin methyl chalcone and palmitoyl tetrapeptide-7 to increase laminin expression. Aspect 24 depends on any of aspects 21 to 23, wherein the topical composition comprises an effective amount of Rosmarinus officinalis leaf extract to inhibit Matrix Metalloproteinase Enzyme Activity. Aspect 25 depends on aspect 24, wherein the Matrix Metalloproteinase Enzyme includes MMP1, MMP3, MMP9, or any combination thereof. Aspect 26 depends on any of aspects 21 to 25, wherein the topical composition comprises an effective amount of Rosmarinus officinalis leaf extract to inhibit a pro-inflammatory cytokine. Aspect 27 depends on aspect 26, wherein the pro-inflammatory cytokine includes lipoxygenase, IL-6, 11-8, TNF-α, FAAH, or any combination thereof. Aspect 28 depends on any of aspects 21 to 27, wherein the topical composition comprises an effective amount of escin to inhibit a pro-inflammatory cytokine including FAAH. Aspect 29 depends on any of aspects 21 to 28, wherein the topical composition comprises an effective amount of Rosmarinus officinalis leaf extract to inhibit elastase. Aspect 30 depends on any of aspects 21 and 29, wherein the topical composition comprises an effective amount of hesperidin methyl chalcone and palmitoyl tetrapeptide-7 to inhibit renin. Aspect 31 depends on any of aspects 21 to 30, wherein the topical composition comprises an effective amount of escin to inhibit melanogenesis. Aspect 32 depends on any of aspects 21 to 31, wherein the topical composition is adapted to provide antioxidant capacity to the skin. Aspect 33 depends on any of aspects 21 to 32, wherein the topical composition is capable of reducing lines and/or wrinkles of skin. Aspect 34 depends on any of aspects 21 to 33, wherein the topical composition is adapted to improve skin firmness. Aspect 35 depends on any of aspects 21 to 34, wherein the topical composition is adapted to reduce dark circles around an eye area. Aspect 36 depends on any of aspects 21 to 35, wherein the topical composition comprises 0.1 to 10 wt. % of a combination of hieracium pilosella extract and bellis perennis flower extract, 0.02 to 2.5 wt. % of lecithin, 0.02 to 1.8 wt. % of escin, 0.005 to 0.5 wt. % hesperidin methyl chalcone, 0.002 to 0.2 wt. % of Rosmarinus officinalis leaf extract, 0.00011 to 0.011 wt. % of plankton extract, 0.00003 to 0.003 wt. % of palmitoyl tetrapeptide-7. Aspect 37 depends on aspect 36, wherein the topical composition further comprises 0.00005 to 0.005 wt. % Opuntia tuna Fruit Extract. Aspect 38 depends on any of aspects 21 to 37, wherein the topical composition further comprises water. Aspect 39 depends on any of aspects 21 to 38, wherein the topical composition is an emulsion, a serum, a gel, a gel emulsion, or a gel serum. Aspect 40 depends on any of aspects 21 to 39, wherein the topical composition is an oil in water emulsion.

“Topical application” means to apply or spread a composition onto the surface of lips or keratinous tissue. “Topical skin composition” includes compositions suitable for topical application on lips or keratinous tissue. Such compositions are typically dermatologically-acceptable in that they do not have undue toxicity, incompatibility, instability, allergic response, and the like, when applied to lips or skin. Topical skin care compositions of the present invention can have a selected viscosity to avoid significant dripping or pooling after application to skin.

“Keratinous tissue” includes keratin-containing layers disposed as the outermost protective covering of mammals and includes, but is not limited to, lips, skin, hair and nails.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase or production of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

As noted above, several of the unique aspects of the present invention are to combine in a topical cosmetic composition containing one or more of escin, lecithin, plankton extract, Hieracium pilosella extract, Bellis perennis flower extract, hesperidin methyl chalcone, palmitoyl tetrapeptide-7, and Rosmarinus officinalis leaf extract. The topical cosmetic composition may further contain Opuntia tuna fruit extract. This allows for the benefits of increasing skin firmness and elasticity, reducing fine lines and/or wrinkles, reducing under-eye puffiness, under-eye dark circles, and sagginess of eyelids, and improving the overall appearance of the area around the eye. Additional benefits include: increasing lysyl oxidase reporter, increasing laminin, inhibiting elastase, inhibiting MMP1, MMP9, and MMP3, inhibiting pro-inflammatory cytokines including lipoxygenase, IL-6, 11-8, TNF-alpha, and FAAH, inhibiting elastatse, inhibiting renin, inhibiting melanogenesis, and providing antioxidant capacity.

The following subsections describe non-limiting aspects of the present invention in further detail.

A particular composition of the present invention is designed to work as an eye cream composition. In one instance, the eye cream can help firm up skin, reduce lines and wrinkles on skin, and increase microcirculation to reduce the appearance of under eye bags while also reducing the appearance of dark circles around an eye. The composition relies on a unique combination of any one of, any combination of, or all of escin, lecithin, plankton extract, Hieracium pilosella extract, Bellis perennis flower extract, hesperidin methyl chalcone, palmitoyl tetrapeptide-7, and Rosmarinus officinalis leaf extract, and further optionally Opuntia tuna fruit extract. An example of such a composition is provided in Example 1, Table 1.

The above compositions can be applied to the skin or hair and remain on the skin or hair for a period of time (e.g., at least 1, 2, 3, 4, 5, 10, 20, 30, or 60 minutes or more). After which, the composition, if needed, can be rinsed from the skin or peeled from the skin. A. Active Ingredients

The present invention is premised on a determination of a combination of active ingredients—escin, lecithin, plankton extract, Hieracium pilosella extract, Bellis perennis flower extract, hesperidin methyl chalcone, palmitoyl tetrapeptide-7, and Rosmarinus officinalis leaf extract—that can effectively increase skin firmness and elasticity, improve skin texture and clarity, reduce fine lines and/or wrinkles, under-eye puffiness, under-eye dark circles, and saggy eyelids, and improve the overall appearance of the area around the eye. Additional benefits include: increasing lysyl oxidase reporter, increasing laminin, inhibiting elastase, MMP1, MMP9, and MMP3, inhibiting pro-inflammatory cytokines including lipoxygenase, IL-6, 11-8, TNF-alpha, FAAH, inhibiting elastatse, inhibiting renin, inhibiting melanogenesis, and providing antioxidant capacity.

This combination of ingredients can be used in different products to treat various skin conditions. By way of example, an eye cream can help firm up skin and improve the overall appearance of the area around the eye while also reducing the appearance of dark circles.

The compositions and formulation of the present invention can be particularly beneficial for skin that has begun to develop lines, wrinkles, and/or sagginess. In addition to counteracting the aging process, the combination of ingredients hydrates, firms, and brightens the skin.

Escin, also known as aescin, is a mixture of saponins found in the seed of Aesculus hippocastanum (the horse chestnut). This active ingredient is commercially available from a variety of sources (see, e.g., International Cosmetic Ingredient Dictionary and Handbook, 12th Edition, 2008 (“CTFA”), Volume 1, page 940, which is incorporated by reference). A mixture of escin and lecithin is available from Indena S.P.A. (Italy) under the trade name Escin β-sitosterol Phytosome.

Plankton extract is an extract obtained from marine biomass. It is known to be a skin conditioner. It may also provide fatty acids, antioxidants, and zinc to the skin as well as reducing inflammation in the skin and protecting the skin from the sun. Plankton extract can be purchased from Lipotec (USA) under the trade name Eyedeline™. In some embodiments, plankton extract is an extract from the microorganism E. crustaceum. It may provide care of the eye contour area by reducing fluid extravasation and increasing the degradation of bilirubin. Additionally, it may be capable of boosting collagen and elastin synthesis and decreases the formation of advanced glycation end products.

As for the combination of Hieracium pilosella extract and Bellis perennis flower extract, such a combination is available from CLR (Germany) under the trade name Juveneye™. This combination is capable of addressing the most important dermatological processes in the formation and persistence of dark circles. It can activate vital processes which allow for the breakdown of heme, which is both dark-colored and plays an important role in the etiology of dark circles. The combination may further be capable of activating dermal draining, through its ability to increase the production of VEGF-C. Excess fluids, electrolytes and immune cells can be drained away more effectively. Additionally, the combination may be able to reduce melanin deposition in the skin and improve overall skin health.

Hesperidin methyl chalcone is a chemical compound that is commercially available from a wide range of sources (see International Cosmetic Ingredient Dictionary and Handbook, 12^{th} edition, volumn 1, page 1146 (2008), which is incorporated by reference). In one aspect, this ingredient can be obtained by using Sederama SAS's (France) mixture sold under the trade name Eyeliss™, which is a combination of water, glycerin, hesperidin methyl chalcone, steareth-20, dipeptide-2 (valyl-tryptophane), and palmitoyl tetrapeptide-7 (Pal-GQPR).

Palmitoyl tetrapeptide-7, which is the reaction product of palmitic acid and a synthetic peptide containing glycine, glutamine, proline, and arginine), is commercially available from a wide range of sources (see International Cosmetic Ingredient Dictionary and Handbook, 12^{th} edition, volumn 2, page 1767 (2008), which is incorporated by reference). In one aspect, this ingredient can be obtained from using Sederama SAS's (France) mixture sold under the trade name Eyeliss™, which is a combination of water, glycerin, hesperidin methyl chalcone, steareth-20, dipeptide-2 (valyl-tryptophane), and palmitoyl tetrapeptide-7 (Pal-GQPR).

Rosmarinus officinalis leaf extract is an extract from the leaf of Rosmarinus officinalis. Rosmarinus officinalis is native to the Mediterranean region, and is a woody, perennial herb with fragrant, evergreen, needle-like leaves and white, pink, purple, or blue flowers. It is a shrub that can reach up to 1.5 meters in height with leaves that are about 2 to 4 cm long with green (top surface) and white (bottom surface) coloring. In a preferred instance, the Rosmarinus officinalis leaf extract can be obtained from the leaf of Rosmarinus officinalis. The leaf can be subjected to a eutectigenesis extraction process using a fluid extraction mixture comprising betaine or hydrated betaine, a hydrogen bond donor compound (e.g., polyols, organic acids, etc.), and water. In particular, the leaf portion can be crushed or macerated and then subjected to the aforementioned eutectic fluid extraction mixture to obtain a eutectic extract. The eutectic extract can then be used in the compositions of the present invention. In some preferred instances, the hydrogen bond donor is an organic acid, preferably lactic acid. Eutectigenesis utilizes eutectic solvents which are mixtures of compounds having melting points lower than those of their constituents taken in isolation. In some instances, Rosmarinus officinalis is commercially available. In some instances, Rosmarinus officinalis can be supplied by Naturex (France) under the trade name Rosemary Eucteys™. It was discovered in the context of the present invention that Rosmarinus officinalis leaf extract can reduce or inhibit contraction of myotubes by reducing or inhibiting an influx of calcium in the myotubes and reducing or preventing the occurrence of an action potential. It was also discovered that Rosmarinus officinalis leaf extract can inhibit or reduce MMP-1, MMP-3, and MMP-9 production or activity in skin cells (e.g., keratinocytes or human dermal fibroblasts).

The extracts described herein can be extracts made through extraction methods known in the art and combinations thereof. Non-limiting examples of extraction methods include the use of liquid-liquid extraction, solid phase extraction, aqueous extraction, ethyl acetate, alcohol, acetone, oil, supercritical carbon dioxide, heat, pressure, pressure drop extraction, ultrasonic extraction, etc. Extracts can be a liquid, solid, dried liquid, re-suspended solid, etc.

### B. Amounts of Ingredients

It is contemplated that the compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### C. Vehicles

The compositions of the present invention can include or be incorporated into all types of vehicles and carriers. The vehicle or carrier can be a pharmaceutically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

### D. Structure

The compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, masks, peels, and ointments. Variations and other structures will be apparent to the skilled artisan and are appropriate for use in the present invention.

### E. Additional Ingredients

In addition to the combination of ingredients disclosed by the inventors, the compositions can also include additional ingredients such as cosmetic ingredients and pharmaceutical active ingredients. Non-limiting examples of these additional ingredients are described in the following subsections.

1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

a. UV Absorption Agents

UV absorption agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidene camphor, and isopentyl 4-methoxycinnamate. Non-limiting examples of physical sunblocks include, kaolin, talc, petrolatum and metal oxides (e.g., titanium dioxide and zinc oxide).

b. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, and mixtures thereof.

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

g. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

h. Thickening Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/vp copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

i. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

2. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### F. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

All of the compositions disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims

Formulations having combinations of active ingredients disclosed herein were prepared as topical skin and/or hari compositions. The formulations represented in Tables 1 and 2 were prepared as emulsions (e.g., an oil-in-water emulsion), particularly as an eye cream.

TABLE 1*

| | % Concentration |
|---|---|
| Ingredient | (by weight) |
| Hieracium pilosella extract and Bellis perennis | 1.00 |
| flower extract | |
| Lecithin | 0.25 |
| Escin | 0.18 |
| hesperidin methyl chalcone | 0.05 |
| Rosmarinus officinalis leaf extract | 0.02 |
| Plankton Extract | 0.0011 |
| Palmitoyl Tetrapeptide-7 | 0.0003 |
| Opuntia tuna Fruit Extract (optional) | 0.0005 |
| Water | 55.97 |
| Dimethicone | 8.70 |
| Glycerin | 4.65 |
| Cetyl Alcohol | 3.60 |
| Isohexadecane | 3.00 |
| Denatured Alcohol | 2.77 |
| Stearic Acid | 2.50 |
| Squalane | 2.00 |
| Caprylyl Methicone | 1.50 |
| Ammonium AcryloyldimethylTaurate/VP | 1.10 |
| Copolymer | |
| Mineral Oil | 1.00 |
| Beeswax | 1.00 |
| Octyl Dodecanol | 1.00 |
| Stearyl Alcohol | 1.00 |
| Silica | 1.00 |
| Ceteareth-25 | 0.99 |
| Butylene Glycol | 0.94 |
| Microcrystalline Wax | 0.90 |
| Triethanolamine | 0.90 |
| Titanium Dioxide | 0.80 |
| Dimethiconol | 0.51 |
| MICA | 0.44 |
| Disodium Ethylene Dicocamide PEG-15 Disulfate | 0.39 |
| Lactic Acid | 0.39 |
| Betaine | 0.34 |
| Diazolidinyl Urea | 0.30 |
| Xanthan Gum | 0.15 |
| Disodium EDTA | 0.10 |
| Paraffin | 0.10 |
| Excipients** | q.s . |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 35% w/w, and preferably between 40 to 60% w/w.

TABLE 2*

| | % Concentration |
|---|---|
| Ingredient | (by weight) |
| Bellis perennis flower extract | 0.65 |
| Hieracium pilosella extract | 0.32 |
| Lecithin | 0.25 |
| Escin | 0.18 |
| hesperidin methyl chalcone | 0.05 |
| Rosmarinus officinalis leaf extract | 0.02 |
| Plankton Extract | 0.0011 |
| Palmitoyl Tetrapeptide-7 | 0.0003 |
| Water | 54.57 |
| Dimethicone | 8.74 |
| Glycerin | 4.63 |
| Cetyl Alcohol | 3.60 |
| Isohexadecane | 3.00 |
| Alcohol | 2.78 |
| Stearic Acid | 2.50 |
| Squalane | 2.00 |
| Caprylyl Methicone | 1.50 |
| Octyldodecanol | 1.50 |
| Ammonium | 1.10 |
| AcryloyldimethylTaurate/VP | |
| Copolymer | |
| Triethanolamine | 1.10 |
| Mineral Oil | 1.00 |
| Beeswax | 1.00 |
| Stearyl Alcohol | 1.00 |
| Silica | 1.00 |
| Ceteareth-25 | 0.99 |
| Butylene Glycol | 0.94 |
| Microcrystalline Wax/Cire Microcristalline | 0.90 |
| Titanium Dioxide | 0.69 |
| Dimethiconol | 0.51 |
| Pentylene Glycol | 0.40 |
| Disodium Ethylene Dicocamide PEG-15 Disulfate | 0.39 |
| Lactic Acid | 0.39 |
| MICA | 0.37 |
| Propanediol | 0.35 |
| Betaine | 0.34 |
| Hydroxyacetophenone | 0.25 |
| Propylene glycol | 0.23 |
| Caprylyl glycol | 0.15 |
| Xanthan Gum | 0.15 |
| Disodium EDTA | 0.10 |
| Ethylhexylglycerin | 0.10 |
| Paraffin | 0.10 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 35% w/w, and preferably between 40 to 60% w/w.

### Example 2

### In Vitro Efficacy of Active Ingredients

The efficacy of the ingredients were determined by the following methods. The following are non-limiting assays that can be used in the context of the present invention. It should be recognized that other testing procedures can be used, including, for example, objective and subjective procedures.

It was determined that a combination of Hieracium pilosella extract and Bellis perennis flower extract provides antioxidant capacity, and increases lysyl oxidase. It was determined that Rosmarinus officinalis leaf extract provides antioxidant capacity, inhibits MMP1, MMP9 and MMP3, inhibits pro-inflammatory cytokines including lipoxygenase, IL-6, 11-8, TNF-α, and FAAH, and inhibits elastase. It was determined that a combination of Hesperidin methyl chalcone and Palmitoyl tetrapeptide-7 provides antioxidant capacity, inhibits laminin, and inhibits renin. It was determined that plankton extract provides antioxidant capacity. It was also determined that escin inhibits pro-inflammatory cytokines including FAAH, and inhibits melanogenesis.

A summary of the quantitative in vitro results is found in Table 3 and the method used to determine the properties of the ingredients are provided below.

TABLE 3

| Assay | Ingredient | Activity |
|---|---|---|
| Antioxidant capacity | a combination of | +98% |
| (TEAC) | extract and Bellis perennis flower | |
| | extract | |
| | Rosmarinus officinalis leaf extract | +52% |
| | a combination of Hesperidin Methyl | +47 |
| | Chalcone and Palmitoyl tetrapeptide-7 | |
| | plankton extract | +98% |
| Lysyl oxidase | combination of Hieracium pilosella | +89% |
| reporter | extract and Bellis perennis flower | |
| | extract | |
| Laminin expression | combination of Hesperidin Methyl | +117% |
| | Chalcone and Palmitoyl tetrapeptide-7 | |
| Inhibition of MMP1 | Rosmarinus officinalis leaf extract | −98% |
| Inhibition of MMP9 | Rosmarinus officinalis leaf extract | −61% |
| Inhibition of MMP3 | Rosmarinus officinalis leaf extract | −40% |
| Inhibition of | Rosmarinus officinalis leaf extract | −54% |
| lipoxygenase | | |
| Inhibition of IL-6 | Rosmarinus officinalis leaf extract | −83% |
| Inhibition of II-8 | Rosmarinus officinalis leaf extract | −98% |
| Inhibition of TNF-α | Rosmarinus officinalis leaf extract | −85% |
| Inhibition of FAAH | Rosmarinus officinalis leaf extract | −50% |
| | escin | −50% |
| Inhibition of elastase | Romnarinus officinalis leaf extract | −54% |
| Inhibition of renin | combination of Hesperidin Methyl | −64% |
| | Chalcone and Palmitoyl tetrapeptide-7 | |
| Inhibition of | Escin | −21.51% |
| Melanogenesis (B16 | | |
| pigmentation assay) | | |

Antioxidant (AO) Assay: a combination of Hieracium pilosella extract and Bellis perennis flower extract, and a combination of hesperidin methyl chalcone and palmitoyl tetrapeptide-7, and plankton extract have been shown to possess antioxidant capacity. The antioxidant system of living organisms includes enzymes such as superoxide dismutase, catalase, and glutathione peroxidase; macromolecules such as albumin, ceruloplasmin, and ferritin; and an array of small molecules, including ascorbic acid, α-tocopherol, β-carotene, reduced glutathione, uric acid, and bilirubin. The sum of endogenous and food-derived antioxidants represents the total antioxidant activity of the extracellular fluid. Cooperation of all the different antioxidants provides greater protection against attack by reactive oxygen or nitrogen radicals, than any single compound alone. Thus, the overall antioxidant capacity may give more relevant biological information compared to that obtained by the measurement of individual components, as it considers the cumulative effect of all antioxidants present in plasma and body fluids. Antioxidant capacity of test compounds and extracts were monitored in an in vitro bioassay that measures the total anti-oxidant capacity of the test compounds and extracts using Anti-Oxidant capacity kit #709001 from Cayman Chemical (Ann Arbor, Mich. USA)

Briefly, Anti-Oxidant capacity kit #709001 from Cayman Chemical (Ann Arbor, Mich. USA) relies on the ability of antioxidants in the samples to inhibit the oxidation of ABTS® (2,2′-azino-di-[3-ethylbenzthiazoline sulphonate]) to ABTS®-+ by metmyoglobin. The capacity of the antioxidants in the sample to prevent ABTS oxidation is compared with that of Trolox, a water-soluble tocopherol analogue, and is quantified as molar Trolox equivalents. The protocol can be followed according to manufacturer recommendations. It was determined that a combination of Hieracium pilosella extract and Bellis perennis flower extract possesses antioxidant capacity of 98% of Trolox, a combination of hesperidin methyl chalcone and palmitoyl tetrapeptide-7 possesses antioxidant capacity of 47% of Trolox, Rosmarinus officinalis leaf extract possesses antioxidant capacity of 52% of Trolox, and plankton extract possesses antioxidant capacity of 98% of Trolox.

Lysyl Oxidase Assay: a combination of Hieracium pilosella extract and Bellis perennis flower extract has been shown to increase lysyl oxidase expression. A lysyl oxidase assay was performed on skin cells (e.g., epidermal keratinocytes, fibroblasts, and/or dermal endothelial cells) to determine the ability of the ingredient listed in Table 1 to stimulate expression of lysyl oxidase in skin. Lysyl oxidase can catalyze crosslinking of elastin and collagens, thereby providing for a more structurally rigid matrix for skin. By increasing expression of lysyl oxidase, increased cross-linking of elastin and collagens can occur, which can be beneficial in reducing the appearance of fine lines, wrinkles, sagging skin, and/or non-elastic skin. It was determined that an effective amount of the a combination of Hieracium pilosella extract and Bellis perennis flower extract increased lysyl oxidase expression by 89%.

Laminin Stimulation Assay—a combination of Hesperidin Methyl Chalcone and Palmitoyl tetrapeptide-7 has been shown to increase laminin expression. Laminin is one of major proteins in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin and fibronectin are two structural glycoproteins located in the DEJ. Considered the glue that holds the cells together, laminin and fibronectin are secreted by dermal fibroblasts to help facilitate intra- and inter-cellular adhesion of the epidermal calls to the DEJ. Collagen is an extracellular matrix protein critical for skin structure. Increased synthesis of collagen helps improve skin firmness and elasticity. Laminin expression was monitored in cultured human fibroblasts using immunefluorescent antibodies directed against each protein in an enzyme linked immuno-sorbant assay (ELISA). It was determined that a combination of Hesperidin Methyl Chalcone and Palmitoyl tetrapeptide-7 increased laminin expression by +117%.

Briefly, laminin expression was monitored by quantifying these proteins in cell supernatants of subconfluent normal human adult epidermal fibroblasts (Cascade Biologics) treated with or without 1.0% final concentration of the test ingredient(s) for 3 days in standard DMEM growth medium with 10% fetal bovine serum (Mediatech) at 37° C. in 10% CO₂. Following incubation, laminin content was measured using immunefluorescent antibodies directed against each protein in an enzyme linked immuno-sorbant assay (ELISA). Measurements were normalized for cellular metabolic activity, as determined by bioconversion of 3-(4,5-dimethylthiazol-2-yl)-5-(3-carboxymethoxyphenyl)-2-(4-sulfophenyl)-2H-tetrazolium (MTS).

Matrix Metalloproteinase 3 and 9 Enzyme Activity (MMP3; MMP9) Assay: Rosmarinus officinalis leaf extract has been shown to inhibit MMP3 and MMP9. This assay is an in vitro matrix metalloprotease (MMP) inhibition assay. MMPs are extracellular proteases that play a role in many normal and disease states by virtue of their broad substrate specificity. MMP3 substrates include collagens, fibronectins, and laminin; while MMP9 substrates include collagen VII, fibronectins and laminin. Using Colorimetric Drug Discovery kits from BioMol International for MMP3 (AK-400) and MMP-9 (AK-410), this assay is designed to measure protease activity of MMPs using a thiopeptide as a chromogenic substrate (Ac-PLG-[2-mercapto-4-methyl-pentanoyl]-LG-0C2H5)5,6. The MMP cleavage site peptide bond was replaced by a thioester bond in the thiopeptide. Hydrolysis of this bond by an MMP produced a sulfhydryl group, which reacts with DTNB [5,5′-dithiobis(2-nitrobenzoic acid), Ellman's reagent] to form 2-nitro-5-thiobenzoic acid, which was detected by its absorbance at 412 nm (c=13,600 M-lcm-1 at pH 6.0 and above 7). Rosmarinus officinalis leaf extract was assayed. It was determined that Rosmarinus officinalis leaf extract inhibits MMP3 by 40% and inhibits MMP9 by 61%.

Matrix Metalloproteinase 1 Enzyme Activity (MMP1) Assay: Rosmarinus officinalis leaf extract has been shown to inhibit MMP1. This is an in vitro matrix metalloprotease (MMP) inhibition assay. MMPs are extracellular proteases that play a role in many normal and disease states by virtue of their broad substrate specificity. MMP1 substrates include collagen IV. The Molecular Probes Enz/Chek Gelatinase/Collagenase Assay kit (#E12055) utilizes a fluorogenic gelatin substrate to detect MMP1 protease activity. Upon proteolytic cleavage, bright green fluorescence was revealed and was monitored using a fluorescent microplate reader to measure enzymatic activity.

The Enz/Chek Gelatinase/Collagenase Assay kit (#E12055) from Invitrogen is designed as an in vitro assay to measure MMP1 enzymatic activity. Rosmarinus officinalis leaf extract was assayed. The assay relies upon the ability of purified MMP1 enzyme to degrade a fluorogenic gelatin substrate. Once the substrate was specifically cleaved by MMP1 bright green fluorescence is revealed and may be monitored using a fluorescent microplate reader. Test materials were incubated in the presence or absence of the purified enzyme and substrate to determine their protease inhibitor capacity. It was determined that Rosmarinus officinalis leaf extract inhibits MMP1 by 98%.

Lipoxygenase (LO) Assay: An in vitro lipoxygenase (LO) inhibition assay. Rosmarinus officinalis leaf extract has been shown to inhibit lipoxygenase. LOs are non-heme iron-containing dioxygenases that catalyze the addition of molecular oxygen to fatty acids. Linoleate and arachidonate are the main substrates for LOs in plants and animals. Arachadonic acid may then be converted to hydroxyeicosotrienenoic (HETE) acid derivatives, that are subsequently converted to leukotrienes, potent inflammatory mediators. This assay provides an accurate and convenient method for screening lipoxygenase inhibitors by measuring the hydroperoxides generated from the incubation of a lipoxygenase (5-, 12-, or 15-LO) with arachidonic acid. The Colorimetric LO Inhibitor screening kit (#760700, Cayman Chemical) was used to determine the ability of Rosmarinus officinalis leaf extract to inhibit enzyme activity. Purified 15-lipoxygenase and Rosmarinus officinalis leaf extract can be mixed in assay buffer and incubated with shaking for 10 min at room temperature. Following incubation, arachidonic acid was an be added to initiate the reaction and the mixture was incubated for an additional 10 min at room temperature. Colorimetric substrate was added to terminate catalysis and color progression was evaluated by fluorescence plate reading at 490 nm. The percent inhibition of lipoxyganse activity was calculated compared to non-treated controls to determine the ability of Rosmarinus officinalis leaf extract to inhibit the activity of purified enzyme. It was determined that Rosmarinus officinalis leaf extract inhibits lipoxygenase by 54%.

Inhibition of Tumor Necrosis Factor Alpha (TNF-α)—Rosmarinus officinalis leaf extract has been shown to inhibit TNF-α production in keratinocytes. TNF-α is the prototype ligand of the TNF superfamily. It is a pleiotropic cytokine that plays a central role in inflammation. Increase in its expression is associated with an up regulation in pro-inflammatory activity. The bioassay used to analyze the effect of Rosmarinus officinalis leaf extract used a spectrophotometric measurement that reflects the presence of TNF-α and cellular viability. It was determined that Rosmarinus officinalis leaf extract inhibits TNF-α production in keratinocytes by 85%.

Subconfluent normal human adult keratinocytes (Cascade Biologics) cultivated in EpiLife standard growth medium (Cascade Biologics) at 37° C. in 5% CO₂, were treated with phorbol 12-myristate 13-acetate (PMA, 10 ng/ml, Sigma Chemical, #P1585-1MG) and either Rosmarinus officinalis leaf extract (treated samples) or no additional treatment (untreated sample) for 6 hours. PMA causes a dramatic increase in TNF-α secretion which peaks at 6 hours after treatment. Following incubation, cell culture medium was collected and the amount of TNF-α secretion quantified using a sandwich enzyme linked immuno-sorbant assay (ELISA) from R&D Systems (#DTA00C).

Briefly, the ELISA assay employed the quantitative sandwich enzyme immunoassay technique whereby a monoclonal antibody specific for TNF-α was been pre-coated onto a microplate. Standards and treated and untreated samples were pipetted into the microplate wells to allow any TNF-α present to be bound by the immobilized antibody. After washing away any unbound substances, an enzyme-linked polyclonal antibody specific for TNF-α was added to the wells. Following a wash to remove any unbound antibody-enzyme reagent, a substrate solution was added to the wells to allow color development in proportion to the amount of TNF-α bound in the initial step. The color development was stopped at a specific time and the intensity of the color at 450 nm was measured using a microplate reader.

Cytokine array (Including IL-6, 11-8, and FAAH): Rosmarinus officinalis leaf extract has been shown to inhibit IL-6, 11-8, and FAAH production. Escin has been shown to inhibit FAAH production. Rosmarinus officinalis leaf extract inhibition of IL-6, 11-8, and FAAH production, and escin inhibition of FAAH production were determined with a protein detection assay using biotinylated antibodies to a variety of cytokines for detection of the antibodies. It was determined that Rosmarinus officinalis leaf extract inhibits IL-6 production by 83%, 11-8 production by 98%, and FAAH production by 50%. It was determined that escin inhibits FAAH production by 50%.

Briefly, human epidermal keratinocytes were cultured to 70-80% confluency. The media in the plate was aspirated and 0.025% trypsin/EDTA was added. When the cells became rounded, the culture dish was gently tapped to release the cells. The trypsin/EDTA containing cells were removed from the culture dish and neutralized. Cells were centrifuged for 5 min. at 180 x g. The cells formed a pellet and the supernatant was aspirated. The resulting pellet was resuspended in EpiLife™ media (Cascade Biologics). The cells were seeded in 6-well plates at approximately 10-20% confluency. After the cells became approximately 80% confluent, the media was aspirated and 1.0 ml of EpiLife™, along with phorbol 13-Myristate 12-acetate (“PMA”) (a known inducer of inflammation) and the test composition dilutions were added to two replicate wells (i.e., 1.0% (100 μl of 100× stock) and 0.1% (10 μl of 100× stock) test compositions were diluted into a final volume of 1 ml EpiLife Growth Medium). The media was gently swirled to ensure adequate mixing. In addition, 1.0 ml of EpiLife™ was added to the control wells, with and without additional PMA. The plates were then incubated at 37±1° C. and 5.0±1% CO₂ for approximately 5 hours after dosing. Following this 5-hour incubation, all media was collected in conical tubes and frozen at −70° C. and the frozen media was subsequently shipped on dry ice.

On the day of the analysis, a 16-pad hybridization chamber was attached to 16-pad FAST slides arrayed in triplicate with 16 anti-cytokine antibodies (including IL-6, 11-8, and FAAH) plus experimental controls (Whatman BioSciences), and the slides were placed into a FASTFrame (4 slides per frame) for processing. Arrays were blocked for 15 min. at room temperature using 70 ml S&S Protein Array Blocking buffer (Whatman Schleicher and Scheull). Blocking buffer was removed and 70 ml of each supernatant sample was added to each array. Arrays were incubated for 3 hours at room temp. with gentle agitation. Arrays were washed 3 times with TBS-T. Arrays were treated with 70 ml of an antibody cocktail, containing one biotinylated antibody corresponding to each of the arrayed capture antibodies. Arrays were incubated for 1 hour at room temp. with gentle agitation. Arrays were washed 3 times with TBS-T. Arrays were incubated with 70 ml of a solution containing streptavidin-Cy5 conjugate for 1 hour at room temp. with gentle agitation. Arrays were washed 3 times with TBS-T, quickly rinsed in de-ionized water, and dried.

Slides were imaged in a Perkin-Elmer ScanArray 4000 confocal fluorescent imaging system. Array images were saved and analyzed using Imaging Research ArrayVision software. Briefly, spot intensities were determined by subtracting background signal. Spot replicates from each sample condition were averaged and then compared to the appropriate controls.

Inhibition of Elastase Activity—Rosmarinus officinalis leaf extract has been shown to inhibit elastase activity. Elastase is an enzyme that degrades elastin. Rosmarinus officinalis leaf extract inhibition of elastase activity was determined using the EnzChek® Elastase Assay (Kit #E-12056) from Molecular Probes (Eugene, Oreg. USA). This kit was used as an in vitro enzyme inhibition assay for measuring inhibition of elastase activity. It was determined that Rosmarinus officinalis leaf extract inhibits elastase activity by 54%.

Briefly, the EnzChek kit contains soluble bovine neck ligament elastin that is labeled with dye such that the conjugate's fluorescence was quenched. The non-fluorescent bovine neck ligament elastin substrate was digested by elastase or other proteases to yield highly fluorescent fragments. The resulting increase in fluorescence was monitored with a fluorescence microplate reader. Digestion products from the elastin substrate have absorption maxima at −505 nm and fluorescence emission maxima at −515 nm. Rosmarinus officinalis leaf extract or no treatment was added into the digestion reaction to determine inhibition of elastase. As a positive control, N-methoxysuccinyl-Ala-Ala-Pro-Val-chloromethyl ketone was used as selective, collective inhibitor of elastase activity.

B16 Pigmentation Assay—Escin has been shown to inhibit melanogenesis.

Melanogenesis is the process by which melanocytes produce melanin, a naturally produced pigment that imparts color to skin, hair, and eyes. Inhibiting melanogenesis is beneficial to prevent skin darkening and lighten dark spots associated with aging. Melanogenesis in B16 cells was determined in the presence or absence of escin by measuring melanin secretion. It was determined that escin inhibits melanogenesis by 21.25%.

Melanogenesis was determined using B16-F1 melanocytes (ATCC), an immortalized mouse melanoma cell line. The endpoint of this assay was a spectrophotometric measurement of melanin production and cellular viability. B16-F1 melanocytes, were cultivated in standard DMEM growth medium with 10% fetal bovine serum (Mediatech) at 37° C. in 10% CO₂ and then treated with or without niacinamide for 6 days. Following incubation, melanin secretion was measured by absorbance at 405 nm and cellular viability was quantified.

### Example 3

(Clinical Efficacy of Compositions)

Anti-Aging and Dark Circles Study—The compositions described in Table 1 and/or Table 2 have been shown to improve saggy eyelids, increase skin firmness, reduce under-eye puffiness, reduce dark circle around an eye area, and reduce lines and wrinkles in the eye area of volunteer subjects. The objective of this study was to assess the effectiveness of the compositions of Tables 1 and 2 on aged/photo-aged skin in the eye area. All the subjects were in age group of 35 to 65 years. The composition was applied to the eye area of each subject twice daily for 12 weeks. Supplemental cleanser and SPF product were also provided. The subjects were evaluated after 4 weeks, 8 weeks, 10 weeks, and/or 12 weeks of using the composition. A summary of the clinical results is shown in Tables 4 and 5.

TABLE 4
Percent of panelists improved for skin conditions

| | 4 weeks | 8 weeks | 10 weeks | 12 weeks |
|---|---|---|---|---|
| Lines | 22% | 33% | — | 44% |
| Firmness | 78% | 96% | — | 81% |
| Dark Circles | 46% | 54% | 54% | — |

TABLE 5
Percent improvement of conditions

| | 4 weeks | 8 weeks | 10 weeks | 12 weeks |
|---|---|---|---|---|
| Lines | NS* | 2% | — | 3% |
| Firmness | 6% | 10% | — | 10% |
| Dark Circles | 7% | 11% | 14% | — |

*NS means not statistically significant?

The results show that, after 4 weeks, 22% subjects showed reduced lines around eye area, 7% subjects showed improvement in saggy eyelids, and 78% subjects showed improvement in skin firmness around eye area. After 8 weeks, 33% subjects showed reduced lines around eye area, 4% subjects showed reduction in saggy eyelids, and 96% subjects showed improvement in skin firmness. After 12 weeks, 44% subjects showed reduced lines around eye area, 4% subjects showed reaction in saggy eyelids, and 81% subjects showed improvement in skin firmness.

Overall, after 8 weeks and 12 weeks, the subjects showed about 2% and 3% reduction in lines, respectively. After 4 weeks, 8 weeks, and 12 weeks, the subjects showed 6%, 10%, and 10% improvement in skin firmness, respectively.

For dark circles study, the subjects were evaluated at 4 weeks, 8 weeks, and 10 weeks. The results show that, after 4 weeks use of the composition, 46% subjects showed reduction in dark circles around eye area. After 8 weeks, 54% subjects showed reduction in dark circles around eye area. After 10 weeks, the percentage of subjects that showed reduction in dark circles around eye area stayed at 54%. Overall, after 4 weeks, the dark circles around eye area of the subjects were reduced by about 7%. After 8 weeks, the dark circles around eye area of the subjects were reduced by about 11%. After 10 weeks, the dark circles around eye area of the subjects were reduced by about 14%. In the clinical study of the topical composition, skin fine lines were evaluated with clinical grading techniques. Evaluations can be made independently by two clinicians and averaged. Skin firmness was measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrated slightly into the skin and resulted in measurements that were dependent upon the properties of the outer layers of the skin, including the stratum corneum and outer epidermis and some of the dermal layers.

Skin Moisture/Hydration Assay: The compositions described in Table 1 and/or Table 2 have been shown to improve skin moisturization. Skin moisture/hydration benefits were measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) were used to assess changes in skin hydration. The unit was calibrated according to instrument instructions for each testing day. A notation of temperature and relative humidity was made. Subjects were evaluated as follows: prior to measurement they equilibrated in a room with defined humidity (e.g., 30-50%) and temperature (e.g., 68-72° C.). Three separate impedance readings were taken on each side of the face, recorded, and averaged. The T5 setting was used on the impedance meter which averages the impedance values of every five seconds application to the face. Changes were reported with statistical variance and significance. Each of the active ingredients, any one of the combination of ingredients, or compositions having said combinations disclosed in the specification were assayed according to this process. All day moisturization percent improvement (tested immediately, 8 hours, and 12 hours after application of the compositions) for using the compositions described in Tables 1 and/or 2 are shown in Table 6.

TABLE 6
Results for All Day Moisturization Percent Improvement

| | Immediate | 8 Hours | 12 Hours |
|---|---|---|---|
| Moisturization | 21% | 12% | 11% |
| Improvement | | | |

Overall, immediately after the application of the compositions shown in Tables 1 and/or 2, the subjects showed about 21% moisturization improvement in skin. After 8 hours and 12 hours, the subjects showed about 12% and 11% improvement in moisturization, respectively

### Example 4

(Additional Assays)

Assays that can be used to determine the efficacy of any one of the ingredients or any combination of ingredients or compositions having said combination of ingredients disclosed throughout the specification and claims can be determined by methods known to those of ordinary skill in the art. The following are non-limiting assays that can be used in the context of the present invention. It should be recognized that other testing procedures can be used, including, for example, objective and subjective procedures.

Collagen Stimulation Assay: Collagen is an extracellular matrix protein critical for skin structure. Increased synthesis of collagen helps improve skin firmness and elasticity. This bioassay can be used to examine the effect of any one of the active ingredients, combination of ingredients, or compositions having said combinations disclosed in the specification on the production of procollagen peptide (a precursor to collagen) by human epidermal fibroblasts. The endpoint of this assay is a spectrophotometric measurement that reflects the presence of procollagen peptide and cellular viability. The assay employs the quantitative sandwich enzyme immunoassay technique whereby a monoclonal antibody specific for procollagen peptide has been pre-coated onto a microplate. Standards and samples can be pipetted into the wells and any procollagen peptide present is bound by the immobilized antibody. After washing away any unbound substances, an enzyme-linked polyclonal antibody specific for procollagen peptide can be added to the wells. Following a wash to remove any unbound antibody-enzyme reagent, a substrate solution can be added to the wells and color develops in proportion to the amount of procollagen peptide bound in the initial step using a microplate reader for detection at 450 nm. The color development can be stopped and the intensity of the color can be measured. Subconfluent normal human adult epidermal fibroblasts (Cascade Biologics) cultivated in standard DMEM growth medium with 10% fetal bovine serum (Mediatech) at 37° C. in 10% CO₂, can be treated with each of the combination of ingredients or compositions having said combinations disclosed in the specification for 3 days. Following incubation, cell culture medium can be collected and the amount of procollagen peptide secretion quantified using a sandwich enzyme linked immuno-sorbant assay (ELISA) from Takara (#MK101).

Laminin and Fibronectin Stimulation Assay: Laminin and fibronectin are major proteins in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin and fibronectin are two structural glycoproteins located in the DEJ. Considered the glue that holds the cells together, laminin and fibronectin are secreted by dermal fibroblasts to help facilitate intra- and inter-cellular adhesion of the epidermal calls to the DEJ. Laminin and fibronectin secretion can be monitored by quantifying laminin and fibronectin in cell supernatants of cultured human fibroblasts treated for 3 days with culture medium with or without 1.0% final concentration of the test ingredient(s). Following incubation, laminin and fibronectin content can be measured using immunofluorescent antibodies directed against each protein in an enzyme linked immuno-sorbant assay (ELISA). Measurements are normalized for cellular metabolic activity, as determined by bioconversion of 3-(4,5-dimethylthiazol-2-yl)-5-(3-carboxymethoxyphenyl)-2-(4-sulfophenyl)-2H-tetrazolium (MTS).

ORAC Assay: Oxygen Radical Absorption (or Absorbance) Capacity (ORAC) of any one of the active ingredients, combination of ingredients, or compositions having said combinations disclosed in the specification can also be assayed by measuring the antioxidant activity of such ingredients or compositions. Antioxidant activity indicates a capability to reduce oxidizing agents (oxidants). This assay quantifies the degree and length of time it takes to inhibit the action of an oxidizing agent, such as oxygen radicals, that are known to cause damage to cells (e.g., skin cells). The ORAC value of any one of the active ingredients, combination of ingredients, or compositions having said combinations disclosed in the specification can be determined by methods known to those of ordinary skill in the art (see U.S. Publication Nos. 2004/0109905 and 2005/0163880; and commercially available kits such as Zen-Bio ORAC Anti-oxidant Assay kit (#AOX-2)). The Zen-Bio ORAC Anti-oxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

Mushroom tyrosinase activity assay: In mammalian cells, tyrosinase catalyzes two steps in the multi-step biosynthesis of melanin pigments from tyrosine (and from the polymerization of dopachrome). Tyrosinase is localized in melanocytes and produces melanin (aromatic quinone compounds) that imparts color to skin, hair, and eyes. Purified mushroom tyrosinase (Sigma) can be incubated with its substrate L-Dopa (Fisher) in the presence or absence of each of the active ingredients, any one of the combination of ingredients, or compositions having said combinations disclosed in the specification. Pigment formation can be evaluated by colorimetric plate reading at 490 nm. The percent inhibition of mushroom tyrosinase activity can be calculated compared to non-treated controls to determine the ability of test ingredients or combinations thereof to inhibit the activity of purified enzyme. Test extract inhibition was compared with that of kojic acid (Sigma).

Cyclooxygenase (COX) Assay: An in vitro cyclooxygenase-1 and -2 (COX-1, -2) inhibition assay. COX is a bifunctional enzyme exhibiting both cyclooxygenase and peroxidase activities. The cyclooxygenase activity converts arachidonic acid to a hydroperoxy endoperoxide (Prostaglandin G2; PGG2) and the peroxidase component reduces the endoperoxide (Prostaglandin H2; PGH2) to the corresponding alcohol, the precursor of prostaglandins, thromboxanes, and prostacyclins. This COX Inhibitor screening assay measures the peroxidase component of cyclooxygenases. The peroxidase activity is assayed colorimetrically by monitoring the appearance of oxidized N,N,N′,N′-tetramethyl-p-phenylenediamine (TMPD). This inhibitor screening assay includes both COX-1 and COX-2 enzymes in order to screen isozyme-specific inhibitors. The Colormetric COX (ovine) Inhibitor screening assay (#760111, Cayman Chemical) can be used to analyze the effects of each of the active ingredients, any one of the combination of ingredients, or compositions having said combinations disclosed in the specification on the activity of purified cyclooxygnase enzyme (COX-1 or COX-2). According to manufacturer instructions, purified enzyme, heme and test extracts can be mixed in assay buffer and incubated with shaking for 15 min at room temperature. Following incubation, arachidonic acid and colorimetric substrate can be added to initiate the reaction. Color progression can be evaluated by colorimetric plate reading at 590 nm. The percent inhibition of COX-1 or COX-2 activity can be calculated compared to non-treated controls to determine the ability of test extracts to inhibit the activity of purified enzyme.

Oil Control Assay: An assay to measure reduction of sebum secretion from sebaceous glands and/or reduction of sebum production from sebaceous glands can be assayed by using standard techniques known to those having ordinary skill in the art. In one instance, the forehead can be used. Each of the active ingredients, any one of the combination of ingredients, or compositions having said combinations disclosed in the specification can be applied to one portion of the forehead once or twice daily for a set period of days (e.g., 1, 2, 3, 4, 5, 6, 7, 8, 9, 10, 11, 12, 13, 14, or more days), while another portion of the forehead is not treated with the composition. After the set period of days expires, then sebum secretion can be assayed by application of fine blotting paper to the treated and untreated forehead skin. This is done by first removing any sebum from the treated and untreated areas with moist and dry cloths. Blotting paper can then be applied to the treated and untreated areas of the forehead, and an elastic band can be placed around the forehead to gently press the blotting paper onto the skin. After 2 hours the blotting papers can be removed, allowed to dry and then transilluminated. Darker blotting paper correlates with more sebum secretion (or lighter blotting paper correlates with reduced sebum secretion.

Erythema Assay: An assay to measure the reduction of skin redness can be evaluated using a Minolta Chromometer. Skin erythema may be induced by applying a 0.2% solution of sodium dodecyl sulfate on the forearm of a subject. The area is protected by an occlusive patch for 24 hrs. After 24 hrs, the patch is removed and the irritation-induced redness can be assessed using the a* values of the Minolta Chroma Meter. The a* value measures changes in skin color in the red region. Immediately after reading, the area is treated with the active ingredients, any one of the combination of ingredients, or compositions having said combinations disclosed in the specification. Repeat measurements can be taken at regular intervals to determine the formula's ability to reduce redness and irritation.

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether each of the active ingredients, any one of the combination of ingredients, or compositions having said combinations disclosed in the specification is inducing irritation. The measurements can be made on each side of the face and averaged, as left and right facial values. Skin clarity can also be measured using the Minolta Meter. The measurement is a combination of the a*, b, and L values of the Minolta Meter and is related to skin brightness, and correlates well with skin smoothness and hydration. Skin reading is taken as above. In one non-limiting aspect, skin clarity can be described as L/C where C is chroma and is defined as (a²+b2)^{1/2}.

Skin Dryness, Surface Fine Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Clinical Grading of Skin Tone Assay: Clinical grading of skin tone can be performed via a ten point analog numerical scale: (10) even skin of uniform, pinkish brown color. No dark, erythremic, or scaly patches upon examination with a hand held magnifying lens. Microtexture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaliness. Skin rough to the touch in some areas; and (1) uneven skin coloration and texture. Numerous areas of scaliness and discoloration, either hypopigmented, erythremic or dark spots. Large areas of uneven color more than 1 cm in diameter. Evaluations were made independently by two clinicians and averaged.

Clinical Grading of Skin Smoothness Assay: Clinical grading of skin smoothness can be analyzed via a ten point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations were made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman et al. (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Skin Firmness Assay with a Hargens Ballistometer: Skin firmness can be measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and are of the replicas covered by wrinkles or fine lines was determined.

Surface Contour of the Skin Assay with a Profilometer/Stylus Method: The surface contour of the skin can be measured by using the profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Rₐ=Standardize roughness; lₘ=the traverse (scan) length; and y=the absolute value of the location of the profile relative to the mean profile height (x-axis).

MELANODERM™ Assay: In other non-limiting aspects, the efficacy of each of the active ingredients, any one of the combination of ingredients, or compositions having said combinations disclosed in the specification can be evaluated by using a skin analog, such as, for example, MELANODERM™. Melanocytes, one of the cells in the skin analog, stain positively when exposed to L-dihydroxyphenyl alanine (L-DOPA), a precursor of melanin. The skin analog, MELANODERM™, can be treated with a variety of bases containing each of the active ingredients, any one of the combination of ingredients, or compositions having said combinations disclosed in the specification or with the base alone as a control. Alternatively, an untreated sample of the skin analog can be used as a control.

Production of Filaggrin: Changes in the production of filaggrin in keratinocytes due to each of the active ingredients, any one of the combination of ingredients, or compositions having said combinations disclosed in the specification can be measured. Filaggrin is the precursor to Natural Moisturizing Factor (NMF) in the skin. Increased NMF increases the moisture content of the skin. Filaggrin production in treated and non-treated keratinocytes can be determined using a bioassay that analyzes filaggrin concentration in keratinocyte cell lysates. A non-limiting example of a bioassay that can be used to quantify filaggrin production is the PROTEINSIMPLE® Simon™ western blotting protocol. For each sample, normal human epidermal keratinocytes (NHEK) are grown in EPI-200—Mattek Epilife® growth media with calcium from Life Technologies (M-EP-500-CA). NHEK are incubated in growth medium overnight at 37° C. in 5% CO₂ prior to treatment. NHEK are then incubated in growth medium with 1% test compound/extract or no compound/extract (negative control) for 24 to 36 hours. The NHEK can then be washed, collected, and stored on ice or colder until lysed on ice using a lysis buffer and sonication. The protein concentrations of the samples can be determined and used to normalize the samples. The lysates can be stored at −80° C. until use in the quantification assay.

The PROTEINSIMPLE® Simon™ western blotting bioassay assay employs a quantitative western blotting immunoassay technique using an antibody specific for filaggrin to quantitatively detect filaggrin in the test samples. Cell samples are lysed and normalized for protein concentration. Normalized samples and molecular weight standards can then be loaded and ran on a denatured protein separation gel using capillary electrophoresis. The proteins in the gel are immobilized and immunoprobed using a primary antibody specific for filaggrin. The immobilized proteins can then be immunoprobed with an enzyme-linked detection antibody that binds the primary antibody. A chemiluminescent substrate solution can then be added to the immobilized proteins to allow chemiluminescent development in proportion to the amount of filaggrin bound in the immobilization. The chemiluminescent development is stopped at a specific time and the intensity of the chemiluminescent signal can be measured and compared to positive and negative controls.

Production of Occludin: Changes in the production of occludin in keratinocytes due to each of the active ingredients, any one of the combination of ingredients, or compositions having said combinations disclosed in the specification can be measured. Occludin is a protein critical to the formulation of tight junctions and the skin's moisture barrier function. A non-limiting example of how occludin production in treated and non-treated keratinocytes can be determined is by the use of a bioassay that analyzes occludin concentration in keratinocyte cell lysates. The bioassay can be performed using PROTEINSIMPLE® Simon™ western blotting protocol. For the samples, adult human epidermal keratinocytes (HEKa) from Life Technologies (C-005-5C) can be grown at 37° C. and 5% CO₂ for 24 hours in Epilife growth media with calcium from Life Technologies (M-EP-500-CA) supplemented with Keratinocyte Growth Supplement (HKGS) from Life Technologies (S-101-5). HEKa are then incubated in growth medium with test compound/extract, no compound/extract for negative control, or with 1 mM CaCl₂) for positive control for 24 to 48 hours. The HEKa are then washed, collected, and stored on ice or colder until lysed on ice using a lysis buffer and sonication. The protein concentrations of the samples can be determined and used to normalize the samples. The lysates are stored at −80° C. until use in the bioassay.

The PROTEINSIMPLE® Simon™ western blotting bioassay assay employs a quantitative western blotting immunoassay technique using an antibody specific for occludin to quantitatively detect occludin in the test samples. Cell samples are lysed and normalized for protein concentration. Normalized samples and molecular weight standards are then loaded and ran on a denatured protein separation gel using capillary electrophoresis. The proteins in the gel are then immobilized and immunoprobed using a primary antibody specific for occludin. The immobilized proteins are immunoprobed with an enzyme-linked detection antibody that binds the primary antibody. A chemiluminescent substrate solution is then added to the immobilized proteins to allow chemiluminescent development in proportion to the amount of occludin bound in the immobilization. The chemiluminescent development can be stopped at a specific time and the intensity of the chemiluminescent signal can be measured and compared to positive and negative controls.

Keratinocyte Monolayer Permeability: Changes in the permeability of a keratinocyte monolayer due to each of the active ingredients, any one of the combination of ingredients, or compositions having said combinations disclosed in the specification can be measured. Keratinocyte monolayer permeability is a measure of skin barrier integrity. Keratinocyte monolayer permeability in treated and non-treated keratinocytes can be determined using, as a non-limiting example, the In Vitro Vascular Permeability assay by Millipore (ECM642). This assay analyzes endothelial cell adsorption, transport, and permeability. Briefly, adult human epidermal keratinocytes from Life Technologies (C-005-5C) can be seeded onto a porous collagen-coated membrane within a collection well. The keratinocytes are then incubated for 24 hours at 37° C. and 5% CO₂ in Epilife growth media with calcium from Life Technologies (M-EP-500-CA) supplemented with Keratinocyte Growth Supplement (HKGS) from Life Technologies (S-101-5). This incubation time allows the cells to form a monolayer and occlude the membrane pores. The media is then replaced with fresh media with (test sample) or without (non-treated control) test compounds/extracts and the keratinocytes are incubated for an additional 48 hours at 37° C. and 5% CO₂. To determine permeability of the keratinocyte monolayer after incubation with/without the test compound/extract, the media is replaced with fresh media containing a high molecular weight Fluorescein isothiocyanate (FITC)-Dextran and the keratinocytes are incubated for 4 hours at 37° C. and 5% CO₂. During the 4 hours incubation, FITC can pass through the keratinocytes monolayer and porous membrane into the collection well at a rate proportional to the monolayer's permeability. After the 4 hour incubation, cell viability and the content of FITC in the collection wells can be determined. For the FITC content, the media in the collection well is collected and fluorescence of the media determined at 480 nm (Em) when excited at 520 nm. Percent permeability and percent change in comparison to the non-treated controls can be determined by the following equations: Percent Permeability=((Mean Ex/Em of test sample)/Mean Ex/Em untreated control)*100; Percent Change=Percent Permeability of test sample—Percent Permeability of untreated control.

Production of Hyaluronic Acid: Changes in the production of hyaluronic acid in human dermal fibroblasts due to each of the active ingredients, any one of the combination of ingredients, or compositions having said combinations disclosed in the specification can be measured. HA is a polysaccharide involved in stabilization of the structure of the matrix and is involved in providing turgor pressure to tissue and cells. As one non-limiting example, HA production in treated and non-treated adult human dermal fibroblasts (HDFa) cells can be determined using the Hyaluronan DuoSet ELISA kit from R&D Systems (DY3614). In this assay, for production of samples, subconfluent HDFa cells from Cascade Biologics (C-13-5C) are incubated at 37° C. and 10% CO₂ in starvation medium (0.15% fetal bovine serum and 1% Penicillin Streptomycin solution in Dulbecco's Modified Eagle Medium) for 72 hours prior to treatment. The cells are then incubated with fresh starvation medium with either test compound, positive control (phorbol 12-myristate 13-acetate from Sigma-Aldrich (P1585) and platelet derived growth factor from Sigma-Aldrich (P3201)), or no additive for 24 hours. Media is then collected and frozen at −80° C. until use in the ELISA assay.

Briefly, the ELISA assay employs a quantitative sandwich enzyme immunoassay technique whereby a capture antibody specific for HA can be pre-coated onto a microplate. Standards and media from treated and untreated cells are pipetted into the microplate wells to enable any HA present to be bound by the immobilized antibody. After washing away any unbound substances, an enzyme-linked detection antibody specific for HA is added to the wells. Following a wash to remove any unbound antibody-enzyme reagent, a substrate solution is added to the wells to allow color development in proportion to the amount of HA bound in the initial step. The color development is stopped at a specific time and the intensity of the color at 450 nm can be measured using a microplate reader.

Inhibition of Hyaluronidase Activity: Changes in the activity of hyaluronidase due to each of the active ingredients, any one of the combination of ingredients, or compositions having said combinations disclosed in the specification can be measured. Hyaluronidase is an enzyme that degrades HA. HA is a polysaccharide involved in stabilization of the structure of the matrix and is involved in providing turgor pressure to tissue and cells. As one non-limiting example, hyaluronidase activity can be determined using an in vitro protocol modified from Sigma-Aldrich protocol #EC 3.2.1.35. Briefly, hyaluronidase type 1-S from Sigma-Aldrich (H3506) is added to microplate reaction wells containing test compound or controls. Tannic acid can be used as a positive control inhibitor, no test compound can be added for the control enzyme, and wells with test compound or positive control but without hyaluronidase can be used as a background negative control. The wells are incubated at 37° C. for 10 minutes before addition of substrate (HA). Substrate is added and the reactions incubated at 37° C. for 45 minutes. A portion of each reaction solution is then transferred to and gently mixed in a solution of sodium acetate and acetic acid pH 3.75 to stop that portion of the reaction (stopped wells). The stopped wells and the reaction wells should both contain the same volume of solution after addition of the portion of the reaction solution to the stopped wells. Both the reaction wells and the stopped wells are incubated for 10 minutes at room temperature. Absorbance at 600 nm is then measured for both the reaction wells and the stopped wells. Inhibition can be calculated using the following formulas: Inhibitor (or control) activity=(Inhibitor stopped wells absorbance at 600 nm—inhibitor reaction wells absorbance at 600 nm); Initial activity=control enzyme absorbance at 600 nm; Percent Inhibition=[(Initial activity/Inhibitor Activity)*100]−100.

Peroxisome Proliferator-Activated Receptor Gamma (PPAR-γ) Activity: Changes in the activity of PPAR-γ due to each of the active ingredients, any one of the combination of ingredients, or compositions having said combinations disclosed in the specification can be measured. PPAR-γ is a receptor critical for the production of sebum. As one non-limiting example, the activity of PPAR-γ can be determined using a bioassay that analyzes the ability of a test compound or composition to inhibit binding of a ligand. Briefly, fluorescent small-molecule pan-PPAR ligand, FLUORMONE™ Pan-PPAR Green, available from Life Technologies (PV4894), can be used to determine if test compounds or compositions are able to inhibit binding of the ligand to PPAR-γ. The samples wells include PPAR-γ and fluorescent ligand and either: test compound or composition (test); a reference inhibitor, rosiglitazone (positive control); or no test compound (negative control). The wells are incubated for a set period of time to allow the ligand opportunity to bind the PPAR-γ. The fluorescence polarization of each sample well can then be measured and compared to the negative control well to determine the percentage of inhibition by the test compound or composition.

Endothelial Tube Formation: Endothelial tube formation is involved in angiogenesis and micro-vessel capillary formation. Capillary formation and angiogenesis may contribute to redness and rosacea of the skin. The ability for endothelial cells to form tubes in the presence or absence of test extracts and compounds may be determined using a capillary tubule disruption assay with pre-formed primary human umbilical vein endothelial cells (HUVEC) in a cell culture system.

Briefly, HUVECs are cultured in vitro on Extracellular Matrix, which stimulates the attachment and tubular morphogenesis of endothelial cells to form capillary-like lumen structures. These in vitro formed capillary tubules are similar to human blood vessel capillaries in many aspects. The capillary tube assay is based on this phenomenon and is used for evaluation of potential vasculature targeting agents.

HUVEC cultures are grown in a 5% CO₂ 37° C. cell incubator. The full growth medium for HUVECs is Endothelial Cell Basal Medium (EBM) supplemented with 2% fetal bovine serum (FBS), 12 μg/ml bovine brain extract, 1 μg/ml hydrocortisone, and 1 μg/ml GA-1000 (gentamicin-amphothericin). HUVEC cultures between passage 3 and 8 may be used for all assay experiments.

HUVECs are pre-labeled with fluorescent agent Calcein AM and seeded in Extracellular Matrix coated 96-well culture plate with their full growth medium. After about four hours of the morphogenesis process, the endothelial capillary tubes should be formed. Then, test agent in designed doses in 50 μl volume is applied into the formed capillary tubule cultures as treatment conditions. The no-treatment controls can be added with vehicle of test agents. Sutent, a FDA approved anti-angiogenic drug one concentration can be included as assay performance control. After about six hours of treatment, the endothelial tubule morphology in each well is examined by microscopy, imaged, and the capillary disrupting activities under treatment conditions can be quantitatively analyzed. Each test conditions can be conducted in duplicate wells, including controls.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Cosmetic Ingredient Dictionary, Third Edition, CTFA, 1982
International Cosmetic Ingredient Dictionary, Fourth edition, CTFA, 1991
International Cosmetic Ingredient Dictionary and Handbook, Tenth Edition, CTFA, 2004
International Cosmetic Ingredient Dictionary and Handbook, Twelfth Edition, CTFA, 2008

1. A method of improving a condition or appearance of skin, the method comprising applying to the skin an effective amount of a topical composition comprising escin, lecithin, plankton extract, Hieracium pilosella extract, Bellis perennis flower extract, hesperidin methyl chalcone, palmitoyl tetrapeptide-7, Rosmarinus officinalis leaf extract, or any combination thereof.

2. The method of claim 1, wherein, by applying the topical composition, the skin is treated to reduce a skin condition comprising skin lines, skin wrinkles, under-eye puffiness, under-eye dark circles, saggy eyelids, saggy skin, or any combination thereof.

3. The method of claim 1, wherein, by applying the topical composition, the skin is treated to improve skin firmness and/or skin elasticity, and/or increase moisture of the skin.

4. The method of claim 1, wherein, by applying the topical composition, the skin is treated to inhibit matrix metalloproteinase enzyme activity, to stimulate laminin production, to inhibit elastase, renin, melanogenesis, or any combination thereof, to increase lysyl oxidase, and/or to inhibit a pro-inflammatory cytokine.

5. The method of claim 4, wherein the matrix metalloproteinase enzyme includes MMP1, MMP3, MMP9, or any combination thereof; and wherein the pro-inflammatory cytokine includes lipoxygenase, IL-6, 11-8, TNF-alpha, FAAH, or any combination thereof.

6. The method of claim 1, wherein the topical composition is adapted to provide antioxidant capacity.

7. The method of claim 1, wherein the topical composition is a cream, an emulsion, a serum, a gel, a gel emulsion, or a gel serum; and wherein the emulsion includes an oil-in-water emulsion.

8. The method of claim 1, wherein the topical composition is applied to skin around an eye area, a fine line on skin, a wrinkle on skin, a dark circle of skin in an eye area, or any combination thereof.

9. The method of claim 1, wherein the topical composition is applied to saggy skin or non-elastic skin.

10. The method of claim 1, wherein, after topical application, the composition remains on the skin for at least 30 minutes, and wherein the topical composition is applied daily for at least 4 weeks.

11. A topical skin composition comprising escin, lecithin, plankton extract, Hieracium pilosella extract, Bellis perennis flower extract, hesperidin methyl chalcone, palmitoyl tetrapeptide-7, Rosmarinus officinalis leaf extract, or any combination thereof.

12. The topical composition of claim 11, wherein the topical composition comprises an effective amount of Hieracium pilosella extract and Bellis perennis flower extract to increase lysyl oxidase, an effective amount of hesperidin methyl chalcone and palmitoyl tetrapeptide-7 to increase laminin expression, an effective amount of Rosmarinus officinalis leaf extract to inhibit Matrix Metalloproteinase Enzyme Activity, an effective amount of Rosmarinus officinalis leaf extract to inhibit a pro-inflammatory cytokine, an effective amount of escin to inhibit a pro-inflammatory cytokine, an effective amount of Rosmarinus officinalis leaf extract to inhibit elastase, an effective amount of hesperidin methyl chalcone and palmitoyl tetrapeptide-7 to inhibit renin, an effective amount of escin to inhibit melanogenesis, or any combination thereof.

13. The topical composition of claim 12, wherein the matrix metalloproteinase enzyme includes MMP1, MMP3, MMP9, or any combination thereof; and wherein the pro-inflammatory cytokine includes lipoxygenase, IL-6, 11-8, TNF-α, FAAH, or any combination thereof,

14. The topical composition of claim 11, wherein the topical composition is adapted to provide antioxidant capacity to the skin.

15. The topical composition of claim 11, wherein the topical composition is adapted to reduce lines and/or wrinkles of skin.

16. The topical composition of claim 11, wherein the topical composition is adapted to improve skin firmness.

17. The topical composition of claim 11, wherein the topical composition is adapted to reduce dark circles around an eye area.

18. The topical composition of claim 11, wherein the topical composition comprises 0.1 to 10 wt. % of a combination of Hieracium pilosella extract and Bellis perennis flower extract, 0.02 to 2.5 wt. % of lecithin, 0.02 to 1.8 wt. % of escin, 0.005 to 0.5 wt. % hesperidin methyl chalcone, 0.002 to 0.2 wt. % of Rosmarinus officinalis leaf extract, 0.00011 to 0.011 wt. % of plankton extract, 0.00003 to 0.003 wt. % of palmitoyl tetrapeptide-7.

19. The topical composition of claim 11, wherein the topical composition is an emulsion, a serum, a gel, a gel emulsion, or a gel serum.

20. The topical composition of claim 11, wherein the topical composition is an oil in water emulsion.
